(19) United States
(12) Patent Application Publication
(10) Pub. No.: US 2023/0381314 A1
(43) Pub. Date: Nov. 30, 2023

(54) ATTENUATED CANCER CELLS AND METHODS RELATED THERETO

(71) Applicant: The regents of the University of California, Oakland, CA (US)

(72) Inventors: Zhen Gu, Los Angeles, CA (US); Tianyuan Ci, Los Angeles, CA (US)

(21) Appl. No.: 18/032,932

(22) PCT Filed: Oct. 20, 2021
(86) PCT No.: PCT/US2021/055814

§ 371 Date: Apr. 20, 2023

Related U.S. Application Data
(60) Provisional application No. 63/094,034, filed on Oct. 20, 2020.

(51) Int. Cl. A61K 39/00; A61K 31/704; C12N 5/09; A61P 35/00

(52) U.S. Cl. CPC A61K 39/4615; A61K 31/704; C12N 5/0693; A61P 35/00; A61K 2039/5152

(57) ABSTRACT

Provided herein are methods and compositions related to cryo-shocked cells useful as therapeutic agents. The cryo-shocked cells may be formulated as a vaccine. Disclosed are methods of preparing dead cryo-shocked cells. Disclosed are also methods of treating or preventing cancer, comprising administering the cryo-shocked cells. Further, methods for delivering a drug to a target tissue of a patient are disclosed.

### RELATED APPLICATIONS

This application claims the benefit of priority to U.S. Provisional Patent Application Ser. No. 63/094,034, filed Oct. 20, 2020, the contents of which are hereby incorporated herein by reference in their entirety.

### BACKGROUND

Acute myeloid leukemia (AML) is a hematological malignancy which has dismal prognosis with the five-year survival of only 30%. The standard-of-care cytoreductive chemotherapy induces AML remission, but disease relapse frequently occurs. Hematopoietic stem cell transplantation (HSCT) in patients who achieve remission after chemotherapy represents the only curative approach so far. However, HSCT is associated with either the lack of suitable hematopoietic stem cell donors or the high risk of transplantation-related mortality. Hence, there is an urgent need to find new strategies for treating AML.

### SUMMARY

In some aspects, disclosed herein is a composition comprising dead cells. The cells may be cryo-shocked cells. For example, the cells may be cryo-shocked in liquid nitrogen, preferably eliminating the pathogenicity of the dead cells. In some embodiments, the dead cells maintain their major structure, the dead cells maintain their chemotaxis towards a specific tissue, and/or the dead cells are loaded with a drug, such as a cancer therapeutic.

The cancer therapeutic may be a chemotherapeutic agent, such as thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, calicheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid, or capecitabine.

In some embodiments, the dead cells effect targeted delivery of the drug toward a target tissue, such as epithelial tissue, connective tissue, bone marrow, or lymphatic system. In some embodiments, the dead cells are dead cancer cells. In some embodiments, the dead cancer cells promote an immune response, and/or the dead cancer cells activate maturation of dendritic cells.

In some embodiments, the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signetring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hairmatrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, Harding-Passey melanoma, juvenile melanoma, lentigo maligna melanoma, malignant melanoma, acrallentiginous melanoma, amelanotic melanoma, benign juvenile melanoma, Cloudman's melanoma, S91 melanoma, nodular melanoma subungal melanoma, and superficial spreading melanoma.

In some aspects, disclosed herein is a vaccine comprising the composition described herein.

In some aspects, disclosed herein is a method of treating or preventing cancer. The method comprises administering the composition described herein. In another aspect, the method comprises administering the vaccine described herein. Numerous embodiments are further provided that can be applied to any aspect of the present invention described herein. For example, in some embodiments, the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signetring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, Harding-Passey melanoma, juvenile melanoma, lentigo maligna melanoma, malignant melanoma, acrallentiginous melanoma, amelanotic melanoma, benign juvenile melanoma, Cloudman's melanoma, S91 melanoma, nodular melanoma subungal melanoma, and superficial spreading melanoma.

In some embodiments, the composition comprises dead cancer cells, which optionally are the same cancer type as the cancer the method is treating.

In some aspects, disclosed herein are methods of preparing dead cryo-shocked cells. The methods may comprise shocking live cells in liquid nitrogen. In some such embodiments, the live cells are immersed in liquid nitrogen for 1-24 hours, e.g., 8-16 hours. In some embodiments, the cryo-shock eliminates the pathogenicity of the dead cells. In some embodiments, the dead cells maintain their major structure, the dead cells maintain their chemotaxis towards a specific tissue, and/or the live cells are loaded with a drug prior to being shocked. In some embodiments, the method further comprises loading the dead cells with a drug, such as a cancer therapeutic. The cancer therapeutic may be a chemotherapeutic agent, such as thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, callcheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid, or capecitabine.

In some embodiments, the dead cells effect targeted delivery of the drug toward a target tissue, such as epithelial tissue, connective tissue, bone marrow, or lymphatic system. In some embodiments, the dead cells are dead cancer cells. In some embodiments, the dead cancer cells promote an immune response or activate maturation of dendritic cells.

In some embodiments, the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signetring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hairmatrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, Harding-Passey melanoma, juvenile melanoma, lentigo maligna melanoma, malignant melanoma, acrallentiginous melanoma, amelanotic melanoma, benign juvenile melanoma, Cloudman's melanoma, S91 melanoma, nodular melanoma subungal melanoma, and superficial spreading melanoma.

In some aspects, disclosed herein are methods for delivering a drug to a target tissue of a patient. The method may comprise administering the pharmaceutical composition described herein.

### DESCRIPTION OF THE FIGURES

FIGS. 1A-1K show characterization of LNT cells. FIG. 1A shows schematic of the procedure to prepare LNT cells. FIG. 1B shows cellular structure of live and LNT C1498 cells. Cell nucleus was stained by Hoechst 33342 and cytoplasm F-actin was stained by AF488-phalloidin. Scale bars, 10 μm. FIG. 1C shows cellular sizes of live and LNT C1498 cells. The cells were captured by confocal microscopy and cellular size were measured by the software Nano Measurer (cell numbers=200). FIG. 1D shows flow cytometry analysis of live and LNT C1498 cells under same voltages. FSC: forward scatter; SSC: side scatter. FIG. 1E shows SEM images of live and LNT cells. Scale bars, 1 μm. FIG. 1F shows cell viability analysis of live and LNT cells by Live/Dead viability kit. Calcein AM: live cells; EthD-1: dead cells. Scale bar, 10 μm. FIG. 1G shows cell viability analysis of live and LNT cells by CCK8 assay (n=6). FIG. 1H shows in vivo proliferation of live and LNT luciferase tagged C1498 cells indicated by the bioluminescence signal (n=5). FIG. 1I shows survival of mice after challenge with live and LNT tumor cells (n=5). Typical flow cytometry images FIG. 1J and DsRed intensities FIG. 1K of peripheral blood 20 days post-challenge with live and LNT DsRed tagged C1498 cells (n=3). Data are presented as means±s.d. in FIGS. 1G, 1K. Statistical significance was calculated via the log-rank (Mantel-Cox) test in FIG. 1I and ordinary one-way ANOVA in FIG. 1K, *P<0.05, **P<0.01, ***P<0.001.

FIGS. 2A-2H show LNT cells as the drug carrier. CXCR4 (FIG. 2A) and CD44 (FIG. 2B) expression of live and LNT C1498 cells analyzed by confocal microscopy (top) and flow cytometry (bottom). Scale bars, 10 μm. FIG. 2C shows fluorescence images of bone isolated 6 h post-injection of cy5.5 labeled live C1498 cells, LNT C1498 cells and paraformaldehyde-fixed C1498 cells. FIG. 2D shows fluorescence intensities of the bone of indicated groups (n=6). FIG. 2E shows typical confocal image of DOX-loaded LNT cells. Scale bar, 10 μm. FIG. 2F shows cumulative release profile of DOX from LNT cell/DOX (n=3). FIG. 2G shows plasma DOX concentration after intravenous injection of free DOX and LNT cell/DOX with DOX dose of 2.5 mg/kg (n=4). FIG. 2H shows bone marrow DOX content 3 h post-administration of the drug (n=3). Data are presented as means±s.d. in FIGS. 2D, 2F, 2G, 2H. Statistical significance was calculated via ordinary one-way ANOVA in FIG. 2D and Student's t-test in FIGS. 2G, 211, *P<0.05, **P<0.01, ***P<0.001.

FIGS. 3A-3I show therapeutic efficacy of LNT cells in AML model. FIG. 3A shows schematic of the treatment model. FIG. 3B shows AML progression in vivo as indicated by bioluminescence signal expressed by luciferase tagged C1498 cells during different treatments (G1: saline; G2: DOX; G3: LNT cell+Adjuvant; G4: LNT cell/DOX+Adjuvant). FIG. 3C shows quantified bioluminescence of different treatment groups. FIG. 3D shows bioluminescence intensity of treated mice on day 21 (n=6). FIG. 3E shows survival of the mice of different treatment groups (n=6). Serum cytokine levels of IFN-γ (FIG. 3F), TNF-α (FIG. 3G) and proportion of peripheral CD3⁺ T cells (FIG. 311) and CD8⁺ T cells (FIG. 3I) on Day 13 (n=6). Data are presented as means±s.d. in FIG. 3D, 3F, 3G, 3H, 3I). Statistical significance was calculated via ordinary one-way ANOVA in FIG. 3D, 3F, 3G, 3H, 3I and log-rank (Mantel-Cox) test in FIG. 3E, *P<0.05, **P<0.01, ***P<0.001.

FIGS. 4A-4H show in vivo prophylactic efficiency of LNT tumor cells. FIG. 4A shows schematic of the treatment model. Bioluminescence images (FIG. 4B) and quantified bioluminescence (FIG. 4C) of the mice pre-immunized with different treatment formulations (G1: saline; G2: Adjuvant; G3: LNT cell+Adjuvant). FIG. 4D shows bioluminescence intensity of treated mice on day 47 (n=5 for G1 and G2 for one mouse died before day 47, n=7 for G3). FIG. 4E shows survival of the mice after tumor challenge (n=6 for G1 and G2, n=7 for G3). FIG. 4F shows serum cytokine levels 3 days post-challenge of live C1498 cells (n=6 for G1 and G2, n=7 for G3). FIG. 4G shows representative flow cytometry images of CD3⁺ T cells (left) and proportion of peripheral CD3⁺ T cells (right) on day 24 (n=6 for G1 and G2, n=7 for G3). FIG. 4H shows representative flow cytometry images of CD8⁺ T cells (left) and corresponding proportion of peripheral CD8⁺ T cells gating on CD3⁺ T cells (right) on day 24 (n=6 for G1 and G2, n=7 for G3). Data are presented as means±s.d. in FIGS. 4D, 4F, 4G, 4H). Statistical significance was calculated via ordinary one-way ANOVA in FIG. 4D, 4F, 4G, 4H and log-rank (Mantel-Cox) test in FIG. 4E, *P<0.05, **P<0.01, ***P<0.001.

FIG. 5 shows SEM images of live and cryo-treated cells. Typical images of live C1498 cells and LNT C1498 cells. Scale bars, 10 μm.

FIG. 6 shows whole-cell protein expression of LNT cells. SDS-PAGE of whole cell lysate proteins from live (left) and LNT C1498 cells (right). The gel was imaged with the Bio-Rad ChemiDoc MP Imaging System using the stain-free gel imaging mode with 5 min UV activation.

FIG. 7 shows CXCR4 expression of live and LNT C1498 cells. The cells were stained with fluorescence-labeled CXCR4 antibody before confocal microscopy analysis. The live C1498 cells were treated with paraformaldehyde and Triton X-100 before staining. Scale bars, 10 μm.

FIG. 8 shows CD44 expression of live and LNT C1498 cells. The cells were stained with fluorescence-labeled CD44 antibody before confocal microscopy analysis. Scale bars, 10 μm.

FIGS. 9A-9B shows in vivo biodistribution of LNT cells. IVIS image of typical organs (FIG. A) and relative fluorescence intensities (FIG. B) of the mice 6 h-post injection of cy5.5-labeled LNT cells. Error bars represent the s.d. (n=3).

FIG. 10 shows schematic of the procedure to prepare DOX-loaded LNT cells.

FIG. 11 shows in vitro cytotoxicity of LNT cell/DOX against C1498 cells. The in vitro cytotoxicity of different formulations was analyzed via MTT assay. Data are presented as means±s.d. (n=3).

FIGS. 12A-12E shows therapeutic efficacy of different treatments in AML model. FIG. 12A shows schematic of the treatment model. FIG. 12B shows bioluminescence images of the mice in response to intravenous injection of saline, LNT cell, free DOX and LNT cell/DOX (DOX 5 mg/kg, LNT cells 1-2×10⁷). FIG. 12C shows quantified bioluminescence of different treatment groups. FIG. 12D shows bioluminescence intensity on day 21. Data are presented as means±s.e.m. (n=6 for saline and LNT cell groups, n=8 for DOX and LNT cell/DOX groups). Statistical significance was calculated via one-way ANOVA (nonparametric), *P<0.05. FIG. 12E shows survival of the mice of different groups (n=6 for saline and LNT cell groups, n=8 for DOX and LNT cell/DOX groups). Statistical significance was calculated via the log-rank (Mantel-Cox) test, *P<0.05, **P<0.01.

FIGS. 13A-13C shows Activation of immune responses of LNT tumor cells. FIG. 13A shows in vitro activation of DCs by LNT C1498 cells. Typical flow cytometry images and surface marker fluorescence intensities of untreated DCs and DCs treated with LNT C1498 cells. Data are presented as means±s.d. (n=6). Statistical significance was calculated via Student's t-test, *P<0.05, **P<0.01, ***P<0.001. FIG. 13B shows proportions of CD3⁺CD8⁺ T cells and CD3⁺CD4⁺ T cells in peripheral leukocytes 5 days post-injection of indicated formulations. Data are presented as means±s.d. (n=7 for LNT cell+Adjuvant, n=6 for other groups). Statistical significance was calculated via ordinary one-way ANOVA, *P<0.05, **P<0.01, ***P<0.001. FIG. 13C shows serum cytokine levels of IFN-γ, TNF-α and IL-6 5 days post-injection of indicated formulations. Data are presented as means±s.d. (n=7 for LNT cell+Adjuvant, n=6 for other groups). Statistical significance was calculated via ordinary one-way ANOVA, *P<0.05, **P<0.01, ***P<0.001.

FIG. 14 shows peripheral proportion of CD3⁺CD8⁺ T cells. Proportion of CD3⁺CD8⁺ T cells on the gate of peripheral leukocytes after challenge of live C1498 cells. The mice were pre-immunized with indicated formulations. Data are presented as means±s.d. (n=6 for Saline and Adjuvant, n=7 for LNT cell+Adjuvant). Statistical significance was calculated via ordinary one-way ANOVA, *P<0.05.

### DETAILED DESCRIPTION OF THE INVENTION

Live cells can be engineered into drug delivery vehicles to leverage their targeting capability and cargo release behavior. Described herein are methods to obtain “dead cells” by shocking live cancer cells in liquid nitrogen to eliminate pathogenicity, while preserving their major structure and chemotaxis towards the lesion site. These cells can be loaded with anticancer agents, to serve as targeted drug-delivery vehicles. In an acute myeloid leukemia (AML) mouse model, the liquid nitrogen-treated AML cells (LNT cells) could effect targeted delivery of chemotherapeutic doxorubicin (DOX) toward the bone marrow. Moreover, LNT cells themselves, with their native tumor-associated antigens, served as a cancer vaccine to promote an immune response that facilitates AML eradication and prolonged the survival of mice significantly. Pre-immunization with LNT cells along with an adjuvant also protected healthy mice from AML cell challenge.

AML originates in the bone marrow and bone marrow creates leukemia-niches that promote leukemia survival. However, sufficient chemotherapeutics to bone marrow is hard to achieve, and higher doses of chemotherapy can also be toxic to normal tissues and induce severe systematic toxicity. Thus, developing targeting delivery systems are valuable for AML therapy. It is, yet, challenging to engineer bone marrow-targeting moieties and bypass the blood-bone marrow barriers, which hampers the feasibility of drug synthetic carries. Leveraging cells' intrinsic properties offers solutions to overcome these challenges. Since AML cells naturally exhibit bone marrow homing capabilities, an approach to directly use AML cells as drug carriers, whilst eliminating their intrinsic pathogenicity, was developed.

Described herein is a liquid nitrogen-based cryo-shocking method to obtain therapeutic dead cells. These cells maintain the intact structure allowing the cells to carry a drug payload, but lose their proliferation ability and pathogenicity. Specifically, cryo-shocked AML cells maintained their bone marrow homing capability, and served as a drug delivery vehicle of doxorubicin (DOX), which is a critical drug used in the induction chemotherapy in AML. In addition, cryo-shocked AML cells act as a cancer vaccine and stimulate an immune response, that in conjunction with chemotherapy to eradicate leukemia. Also, pre-immunization with LNT cells together with an adjuvant could effectively protect healthy mice from AML cell challenge. Compared to the live cell-mediated drug delivery systems, this “dead cell”-based delivery vehicle can be readily prepared with flexibility associated with cell viability and stability during manufacturing.

The feasibility, safety and efficacy of utilizing “dead cells” as a drug targeting carrier and tumor vaccine for cancer therapy is described herein. Compared to the synthetic materials-mediated delivery vehicles, cells' intrinsic properties could bypass biological barriers and enable the cells of unique targeting capacities. AML cells originate in the bone marrow and naturally exhibit similar bone marrow homing capabilities as HSCs, rendering its potency as cellular drug carriers for enhancing AML therapy. However, strategies to eliminate their pathogenicity but preserving the targeting capacities of live cells are essential.

Usually, the structure of the live cells can disintegrate upon dying with the loss of proteins and cytokines. And external stimuli that could induce cell death, such as heat or radiation, will deactivate proteins as well. We then conceived to apply the cryo-treating process to obtain dead cells. Through a simple modified process based on immersing live cells in liquid nitrogen for storage purpose, the cryo-shocked cell was confirmed to retain its intact cellular structure, which is the basis for the drug loading and cargo release. Furthermore, the two important adhesion receptors of CXCR4 and CD44, that mediate live AML cells toward bone marrow, remained in LNT AML cells. This point was confirmed via analysis of confocal microscopy and flow cytometry after staining LNT cells with specific antibodies. More importantly, this straightforward method to prepare therapeutic dead cells is capable of large-scale production and reproducibility, based on a facile procedure without the kinds of complex quality control typically associated with live cells.

For the perspective of safety, we evaluated the proliferation behavior and tumorigenicity of LNT tumor cells both in vitro and in vivo. All the mice treated with LNT cells exhibited no obvious side effects and no mice died even after 6 months after challenge with LNT C1498 cells. After treatment with liquid nitrogen, the cellular membrane of LNT cells becomes permeable, which was verified by the different staining process between live and LNT cells. Live cells need to be treated with Triton X-100, a cell membrane detergent, before intracellular staining is observed. However, for LNT cells, this step is unnecessary.

In summary, LNT tumor cells were engineered to serve simultaneously as a drug delivery carrier and cancer vaccine. The simple liquid nitrogen treating process abrogates the tumorigenicity of tumor cells, but preserves the integrity of their cellular structure. This in turn allows the possibility to load LNT cells with chemotherapy drugs and preserves the homing capacity of these cells to the tumor site. Furthermore, LNT cells in combination with adjuvant could elicit both therapeutic and protective immune antitumor responses.

### Definitions

For convenience, certain terms employed in the specification, examples, and appended claims are collected here.

As used herein, the singular forms “a”, “an,” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.” The transitional terms “consist” and any grammatical variations thereof, are intended to be limited to the elements stated in the claims and exclude any elements not stated in the claims. The phrases “consisting essentially of” and any grammatical variant thereof indicate that the claim encompasses embodiments containing the specified elements and includes additional elements that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system, and the conventional variability accepted in the art for the concerned parameter.

As used herein, the term “administering” means providing a therapeutic agent or composition to a subject, and includes, but is not limited to, administering by a medical professional and self-administering. The means of providing a therapeutic agent are well known to those skilled in the art and include, but are not limited to, oral administration, transdermal administration, administration by inhalation, nasal administration, topical administration, intravaginal administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “preventing” is art-recognized, and when used in relation to a condition is well understood in the art, and includes administration of a composition which reduces the frequency of, or delays the onset of, symptoms of a medical condition in a subject relative to a subject which does not receive the composition. Thus, prevention of cancer includes, for example, reducing the incidence, number, and/or size of cancer cells in a population of patients receiving a prophylactic treatment relative to an untreated control population, and/or delaying the appearance of scars in a treated population versus an untreated control population, e.g., by a statistically and/or clinically significant amount.

In certain embodiments, a therapeutic agent may be used alone or conjointly administered with another therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, conjoint administration of the combinations of compositions of the invention with one or more additional therapeutic agent(s) (e.g., one or more additional chemotherapeutic agent(s)) provides improved efficacy relative to each individual administration of the combinations of compounds of the invention or the one or more additional therapeutic agent(s). In certain such embodiments, the conjoint administration provides an additive effect, wherein an additive effect refers to the sum of each of the effects of individual administration of the combinations of compositions of the invention and the one or more additional therapeutic agent(s).

The term “a small molecule” is a compound having a molecular weight of less than 2000 Daltons, preferably less than 1000 Daltons. Typically, a small molecule therapeutic is an organic compound that may help regulate a biological process.

“Subject” refers to an animal, such as a mammal, for example a human. The methods described herein can be useful in both humans and non-human animals. In some embodiments, the subject is a mammal (such as an animal model of disease), and in some embodiments, the subject is human.

As used herein, the term “cryo-shocked cells” means cells that were killed or attenuated by immersing them in liquid nitrogen.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the cell is preferably administered as a pharmaceutical composition comprising, for example, the combination of cells described herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration.

The methods of administering cells to a subject as described herein involve the use of therapeutic compositions comprising such cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to, into, or upon a mammal without the production of undesirable physiological effects such as nausea, dizziness, gastric upset, transplant rejection, allergic reaction, and the like. The preparation of a composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared.

In general, the cells described herein are administered as a suspension with a pharmaceutically acceptable carrier. A formulation comprising cells can include, for example, osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

A cell composition can also be emulsified or presented as a liposome composition. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop.

Actual dosage levels of the active ingredients in the pharmaceutical compositions may be varied to obtain an amount of the active ingredient that is effective to achieve the desired therapeutic response for a particular patient, composition, and mode of administration, without being toxic to the patient.

A physician or veterinarian having ordinary skill in the art can readily determine and prescribe the therapeutically effective amount of the pharmaceutical composition required.

For example, the physician or veterinarian could start doses of the pharmaceutical composition or compound at levels lower than that required to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of cells and/or cells loaded with active compounds used in the compositions and methods of the invention will be that amount of the cells or the compounds that is the lowest dose effective to produce a therapeutic effect. Such an effective dose will generally depend upon the factors described above.

If desired, the effective daily dose of the cells may be administered as one, two, three, four, five, six or more sub-doses administered separately at appropriate intervals throughout the day, optionally, in unit dosage forms. In certain embodiments of the present invention, the cells may be administered two or three times daily. In preferred embodiments, the cells will be administered once daily.

### Methods of Treatment

Provided herein are methods of preventing or treating a disease (e.g., χανχερ) comprising administering a composition or a vaccine described herein. The composition or the vaccine comprise dead cells, wherein the cells are cryo-shocked cells, preferably the cells are cryo-shocked in liquid nitrogen. In some embodiments, the cryo-shock eliminates the pathogenicity of the dead cells. In some embodiments, the dead cells maintain their major structure and/or maintain their chemotaxis towards a specific tissue.

In some embodiments, the dead cells are loaded with a drug. For example, the drug may be a cancer therapeutic, such as a chemotherapeutic agent. Examples of the chemotherapeutic agent may be thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, calicheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid, or capecitabine.

In some embodiments, the dead cells augment targeted delivery of the drug toward a specific tissue. The specific tissue may be epithelial tissue, connective tissue, bone marrow, or lymphatic system.

In some embodiments, the dead cells are dead cancer cells. In some embodiments, the dead cancer cells promote an immune response and/or activate maturation of dendritic cells. In some embodiments, the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

### EXAMPLES

The invention now being generally described, it will be more readily understood by reference to the following examples, which are included merely for purposes of illustration of certain aspects and embodiments of the present invention, and are not intended to limit the invention.

### Example 1: Materials and Methods

### Experimental Design

The aim of this study was to utilize the cryo-shocked tumor cells as a kind of drug targeting carrier and tumor vaccine for chemo-immunotherapy in treatment of acute myeloid leukemia. After treating the live cells in liquid nitrogen, the cellular structure of the cryo-shocked cells was observed. The proliferation behavior, in vivo tumorigenicity and targeting capability toward bone marrow of the cryo-shocked cells were assessed. In vivo antitumor efficacy was analyzed in an AML model by intravenously injecting C1498 cells in C57BL/6J mice. Mice were randomly assigned to groups based on body weights. After different treatments, the mice were captured by IVIS to evaluate in vivo tumor progression. Survival curves, immune cell proportions and cytokine levels were determined according to previous experimental experience. Specific information about treatment groups, sample numbers and data analysis were denoted in figure captions.

### Materials, Cell Lines, and Animals

Doxorubicin hydrochloride was purchased from Fisher Scientific Co. (D4193, purity >95%). Noncontrolled-rate cell cryopreservation medium was bought from Cyagen Co. (NCRC-10001-50). Acute myeloid leukemia cell line C1498 was purchased from American Type Culture Collection (ATCC). The cells were cultured in 90% Dulbecco's modified Eagle medium (DMEM, Gibco) and 10% fetal bovine serum (FBS, Gibco) with 200 U mL⁻¹ penicillin and 200 U mL⁻¹ streptomycin (Gibco). The cells were passaged every 1-2 days. C57BL/6J mice (4-6 weeks, female) were purchased from the Jackson laboratory. All animal tests complied with the animal protocol approved by the Institutional Animal Care and Use Committee of the University of California, Los Angeles.

### Preparation of LNT Cells and Drug-Loaded LNT Cells

C1498 cells were centrifuged at 250 g for 3 min and suspended in noncontrolled-rate cell cryopreservation medium at a cell density of 1×10⁶−1×10⁷/mL. The cell-containing medium was immersed in liquid nitrogen for 12 h. Before use, the medium was thawed at 37° C. and LNT cells were pelleted at 500 g for 3 min. After washing with phosphate buffered saline solution (PBS, pH 7.4), LNT cells were suspended in PBS and kept at 4° C. For preparation of DOX-loaded LNT cells, the LNT cells were suspended in DOX containing PBS. After incubation for 2 h, the medium was centrifuged at 500 g for 5 min and the pellets were DOX-loaded LNT cells.

### In Vivo Treatment of AML

AML model was established by intravenous injection of 5×10⁶ C1498 cells on day 0. On day 8 and day 15, saline, LNT cell+Adjuvant, free DOX and LNT cell/DOX+Adjuvant were administrated intravenously with DOX dose of 5 mg/kg and adjuvant (monophosphoryl lipid A, MPLA) 20 μg per mouse. Specifically, MPLA was intravenously injected 10 h post-injection of LNT cell or LNT cell/DOX. The bioluminescence images of mice were captured every 3 days. The exposure time was 2 min. On day 13, 400 μL blood was collected via the orbital vein. 200 μL blood was treated with ACK buffer and centrifuged at 800 g for 8 min to obtain pellets of white blood cells. After staining with BV421-CD3, PE-CD4 and APC-CD8, the samples were analyzed by flow cytometry. Another 200 μL blood in blood serum collection tubes (BD Microtainer 365967) was centrifuged at 3000 rpm for 10 min. The upper serum was detected with the following ELISA kits: IFN-γ (BioLegend 430804) and TNF-α (BioLegend 430904).

### Statistical Analysis

The results were presented as means±s.d. or mean±s.e.m. as indicated. The data were compared by Student's t-test between two groups and ordinary one-way ANOVA for three or more groups. The survival curves were analyzed via the log-rank (Mantel—Cox) test. All statistical analysis was conducted by the GraphPad Prism Software. The threshold of a statistically significant difference was defined as P<0.05.

### Characterization of LNT Cells

The LNT cell structure was analyzed via fluorescence staining with Hoechst (Invitrogen) and AF488 conjugated phalloidin (Invitrogen). Briefly, 1×10⁶ LNT cells were suspended in 1 mL PBS. 25 μL phalloidin stock solution (6.6 μM) was added and the cells were stained at room temperature for 20 min. After that, the cells were centrifuged at 500 g for 3 min and washed with PBS. After the cells re-suspending in 1 mL PBS, 10 μL Hoechst stock solution (10 mg/mL) was added and stained the cells for 10 min. After washing with PBS, the cells were suspended in 500 μL PBS and analyzed by confocal microscopy (Zeiss LSM 880). The live C1498 cells were first fixed with 4% paraformaldehyde (Thermo Scientific) for 15 min and treated with 0.1% Triton X-100 (Thermo Fisher Scientific) for 15 min. The following staining process was similar with LNT cells.

For cell viability analysis, the cells were stained with Live/Dead viability kit (ThermoFisher Scientific #L3224) according to the manufacturer's protocol. After staining, the cells were analyzed by confocal microscopy. In addition, about 200 cells were captured and the cellular size was measured with the Nano Measurer software.

For scanning electron microscopy (SEM) characterization, the cells were fixed in 3.5% glutaraldehyde for 4 hours. After washing with 0.1 M sodium cacodylate buffer (Electron Microscopy Sciences) three times, the cells were fixed for 1 h with 1% osmium tetroxide (Electron Microscopy Sciences). After washing with 0.1 M sodium cacodylate buffer, the cells were dehydrated with graded ethanol (30%, 50%, 70%, 85%, 90% once for 15 min, and 100% twice for 30 min). The cells suspended in 100% ethanol were dropped on silicon. After drying, the silicon was coated by a thin layer of gold and analyzed by SEM (Zeiss Supra 40VP).

### Cell Proliferation of LNT Cells

For in vitro cell proliferation, both live cells and LNT cells were suspended in the cell culture medium (DMEM, no phenol red, 10% FBS) and added to 96-well plates with a cell density of 8×10³ per well. After culturing for 0.5 h, 24 h, 48 h and 72 h, 10 μL cell counting kit-8 solution (CCK-8, Sigma-Aldrich) was added to each well. After incubation for 3 h, the absorbance was measured at 450 nm using a microplate reader (Tecan).

For in vivo cell proliferation, 2×10⁶ live or LNT luciferase and DsRed tagged C1498 cells were injected into the mice intravenously. The proliferation of cells was monitored by detecting the bioluminescence signal at day 7, day 14 and day 21. After 10 min of the intraperitoneal injection of the substrate D-Luciferin (150 mg/kg), the mice were imaged with the IVIS Spectrum Imaging System (PerkinElmer). At day 20, 200 μL blood was collected through the orbital vein. After treatment with ACK buffer (Gibco), the remaining cells were centrifuged at 800 g for 10 min. After suspension in PBS, the cells were analyzed by flow cytometry (BD LSRII). The fluorescence signal of DsRed was recorded.

### Protein Expression of LNT Cells

Whole-cell protein expression was analyzed by SDS-PAGE (sodium dodecyl sulfate-polyacrylamide gel electrophoresis). The proteins were extracted from live and LNT C1498 cells by using RIPA lysis and extraction buffer (ThermoFisher) with protease inhibitor cocktail added (ThermoFisher). Protein concentration was determined by BCA assay (ThermoFisher) according to the manufacturer's instructions. Loading samples were prepared using Laemmli sample buffer (Bio-Rad) with the protein amount of 20 μg per well. After the proteins in the loading samples were denatured for 10 minutes at 95° C., the loading samples were analyzed by SDS-PAGE in a Stain-Free™ Precast Gel (Bio-Rad #4568094). The gel was imaged with the Bio-Rad ChemiDoc MP Imaging System using the stain-free gel imaging mode with 5 min UV activation.

The expression of CD44 and CXCR4 of the cells was analyzed by confocal microscopy and flow cytometry. For CD44, both live and LNT cells were suspended in cell staining buffer (Biolegend) and stained with APC-CD44 for 1 h. After centrifugation and re-suspension in PBS, the cells were imaged by confocal microscopy and analyzed by flow cytometry. For CXCR4, the live cells were first fixed with 4% paraformaldehyde for 15 min and treated with 0.1% Triton X-100 in PBS for 15 min, then suspended in the cell staining buffer and stained with APC-CXCR4 for 1 h. The LNT cells were stained with same process but without treatment with paraformaldehyde and Triton X-100.

### In Vivo Biodistribution of LNT Cells

Live cells and LNT cells were first incubated in cy5.5-NHS (Lumiprobe) containing PBS for 0.5 h to obtain cy5.5 labeled cells. In addition, some of the cy5.5-labeled live C1498 cells were treated with 4% paraformaldehyde for 1 h to denature proteins as the control group. The cy5.5-labeled live and LNT C1498 cells as well as cy5.5-labeled paraformaldehyde-fixed C1498 cells were intravenously injected into the mice with cy5.5 dose of 30 nmol/kg. Six hours later, the mice were euthanized and the organs of heart, liver, spleen, lung, kidneys and the hind limb bones were isolated for fluorescence imaging by IVIS imaging system (Perkin Elmer).

### Characterization of DOX-Loaded LNT Cells

The drug release profile of DOX from LNT cell/DOX was determined. Briefly, 1 mL releasing medium of PBS was added in the well of 12-well plate equipped with 3 Transwell, and 200 μL LNT cell/DOX was added in the chamber of Transwell. The plate was kept in 37° C. incubator (Corning LSE Shaking Incubator) with a shaking rate of 120 rpm. At specified time points, 1 mL of the releasing medium in the well was withdrawn and refreshed with same volume PBS. The DOX concentration was determined by a microplate reader with excitation and emission wavelengths of 480 nm and 598 nm, respectively.

The in vitro cytotoxicity of LNT cell/DOX was determined via MTT assay. Briefly, C1498 cells were cultured in 24-well plate equipped with 1 μm Transwell with a cell density of 2×10⁶ per well. And LNT cell/DOX solution with different DOX concentrations was added in the chamber of Transwell. 24 h later, the Transwell was discarded and 80 μL MTT solution (5 mg/mL) was added to each well. The cells were incubated for further 4 h at 37° C. The upper medium was gently aspirated and 600 μL DMSO was added to dissolve the formed formazan. OD value was detected at 490 nm. IC₅₀ values were analyzed by Graphpad Prism 7.0.

The pharmacokinetics of DOX was monitored after intravenous injection of free DOX and LNT cell/DOX (DOX 2.5 mg/kg). At time points, 150 μL blood was collected via the orbital vein and centrifuged at 5000 rpm for 10 min to get the plasma. 100 μL cold acetonitrile was added to 50 μL plasma and the mixture was centrifuged at 10000 rpm for 10 min to eliminate proteins. The supernatant was withdrawn and detected with fluorescence detector (Tecan Inifinite M Plex).

For DOX accumulation in the bone marrow, the femur and tibia bones of the mice were carefully isolated 3 h post-administration of free DOX and LNT cell/DOX (DOX 2.5 mg/kg), and the bone marrow was flushed with 300 μL DMSO. After centrifugation and filtration with 0.22 μm filter, the sample was analyzed by high performance liquid chromatography (HPLC) equipped with a reverse-phase column of 5 μm C18 (150 mm×4.6 mm, Inertsil ODS-3). The mobile phase was composed of 20.5% acetonitrile, 20% methanol and 59.5% 0.2 M NaH₂PO₄ (v/v/v, pH 4.0). The detection wavelength was set as 480 nm.

### Activation of Dendritic Cells

Bone marrow dendritic cells (BMDC) were collected from the femur and tibia of the mice. Briefly, after the mice were euthanized, the femur and tibia were harvested. Both ends of each bone were cut open, and the bone marrow was flushed with cell culture medium. The cells were first pelleted at 600 g for 5 min and suspended in 3 mL ACK buffer for 3 min. After centrifugation, the cells were washed with PBS twice. Then the cells were cultured in RPMI-1640 medium (10% FBS) with granulocyte/macrophage-colony stimulating factor (GM-CSF, 20 ng/mL, R&D Systems) and IL4 (5 ng/mL, Biolegend) for 7 days. The medium was changed every three days. At day 6, the cells were collected with the cell scraper and cultured in 6-well plates at a cell density of 1×10⁶. At day 7, 1×10⁶ LNT C1498 cells were added to the well without changing the medium. The group of blank medium without LNT cells was set as control. 48 h later, the cells were collected. After suspending in cell staining buffer (Biolegend), the cells were stained with BV421-CD11c, PE-CD80, APC-CD86, APC-CD40 and PE-MHC-II.

### In Vivo Treatment of AML

AML model was established by intravenous injection of 5×10⁶ C1498 cells on day 0. On day 7, day 11 and day 17, saline, LNT cell, free DOX and LNT cell/DOX were administrated intravenously, with DOX dose of 5 mg/kg. The bioluminescence images of mice were captured every 3 days with IVIS imaging system (Perkin Elmer) after 10 min of the intraperitoneal injection of D-Luciferin (150 mg/kg). The exposure time was 2 min.

### In Vivo Prophylactic Efficiency Against AML

Different groups of saline, LNT cell, LNT cell+Adjuvant were intravenously injected at day 0, day 7 and day 14 (LNT cell 5×10⁶ per mouse, MPLA 20 μg per mouse). On day 21, 1×10⁶ live C1498 cells were intravenously injected into the mice. The tumor growth was monitored via bioluminescence intensity by IVIS after 10 min of the injection of D-Luciferin (150 mg/kg). The exposure time was 2 min. At day 5 and day 24, 400 μL, blood was collected via the orbital vein. 200 μL blood was treated with ACK buffer and centrifuged at 800 g for 8 min to get the pellets of white blood cells. After washing with PBS and suspended in cell staining buffer (Biolegend), the cells were stained with BV421-CD3, PE-CD4, and APC-CD8. 200 μL blood was collected in blood serum collection tubes (BD Microtainer 365967) and centrifuged at 3000 rpm for 10 min. The serum was detected with the following ELISA kits: IFN-γ (BioLegend 430804), TNF-α (BioLegend 430904), IL-12 (BioLegend 433604) and IL-6 (BioLegend 431304).

### Example 2: Engineering and Characterization of Liquid Nitrogen-Treated Cells

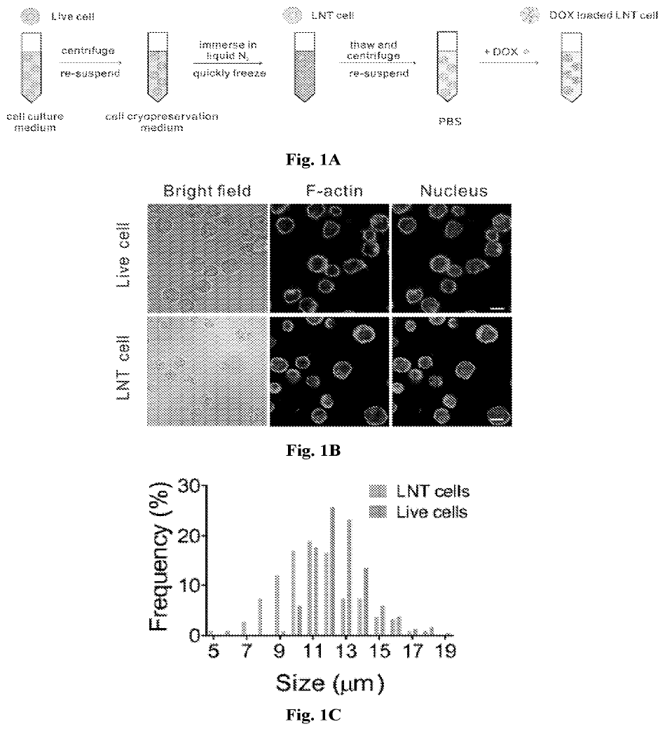

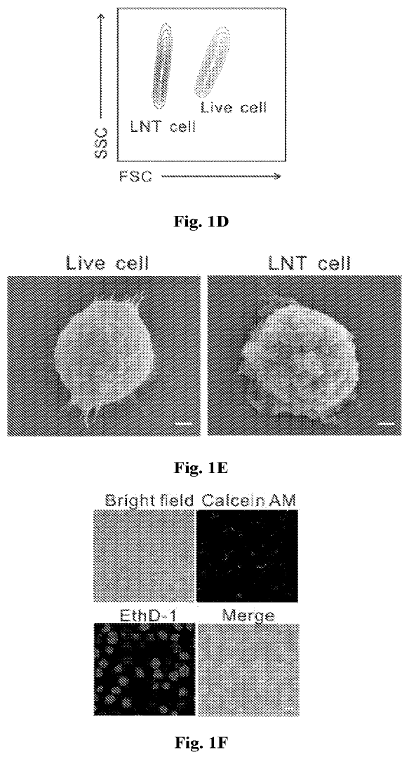

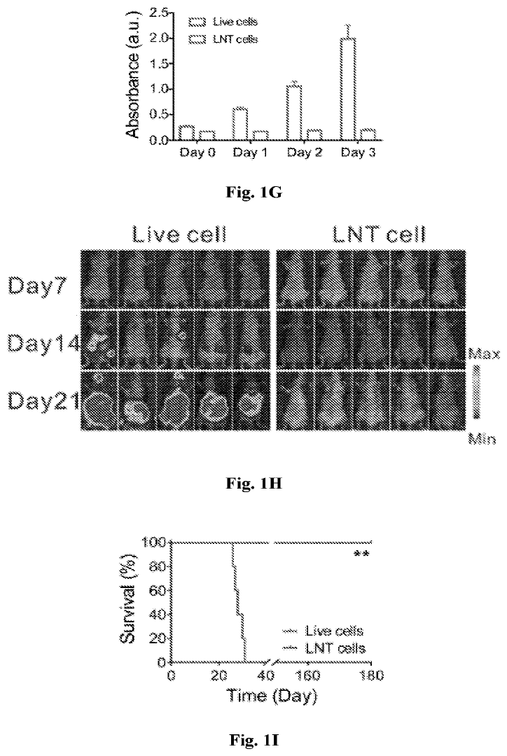

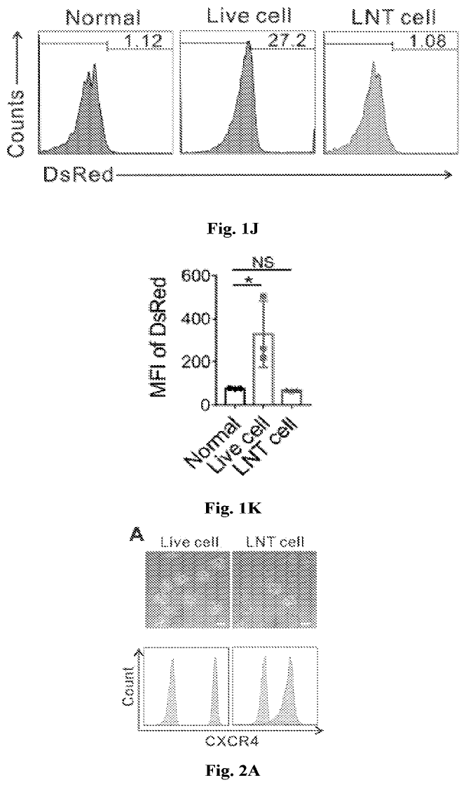

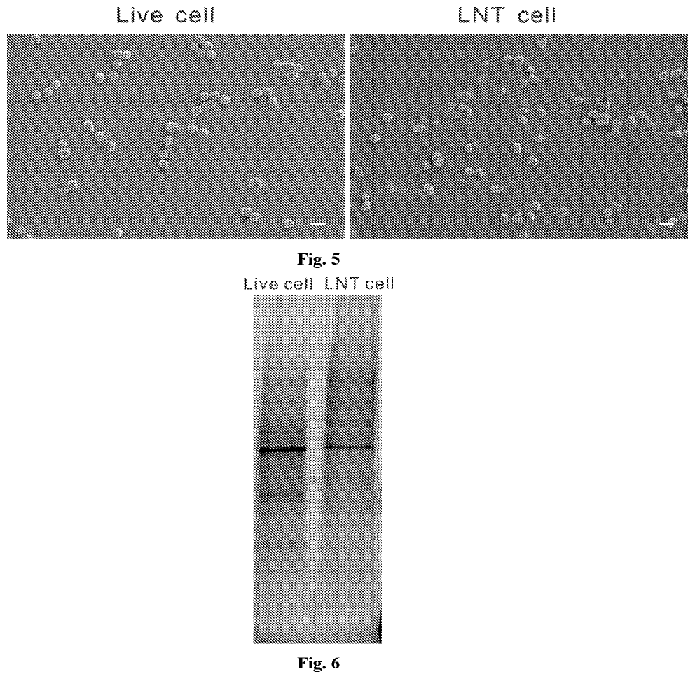

To obtain liquid nitrogen-treated (LNT) cells, AML cells were suspended in cell cryopreservation medium and immersed in liquid nitrogen for 12 hours. LNT cells were then thawed at 37° C. and washed with PBS (FIG. 1A). When analyzed by confocal images, LNT cells showed the same cellular structure of untreated live cells as assessed by nucleus and cytoskeleton staining (FIG. 1B). A slight decrease in cellular size was observed (FIG. 1C), with an average size of 11 μm for LNT cells and 12 μm for untreated live cells. The forward scatter (FSC) values measured by flow cytometry corroborated the cell size reduction of LNT cells, and similar side scatter (SSC) values suggested that the internal structure of LNT cells was maintained (FIG. 1D). Scanning electron microscopy (SEM) images revealed the sphere-like structure of LNT cells and the rougher cellular surface as compared to control live cells (FIG. 1E, FIG. 5).

Next, cell viability of LNT cells was evaluated. As shown in FIG. 1F, nearly all the LNT cells were labeled with EthD-1 (indicating dead cells), and did not show intact fluorescence signal of calcein AM (indicating live cells). Additionally, LNT cells did not show proliferative activity as compared to live cancer cells as measured with counting kit-8 (CCK8) assay (FIG. 1G). The absence of pathogenicity of LNT cells in vivo was demonstrated. As shown in FIG. 1H, live C1498 AML, cells quickly proliferated in mice and caused 100% death in 31 days, while mice receiving C1498 LNT cells exhibited no detectable bioluminescence signal and all mice survived for 180 days (FIGS. 1H, FIG. 1I). Moreover, we quantitatively analyzed cancer cells in the peripheral blood at day 20 post-injection. A significantly higher DsRed signal was observed in mice injected with live C1498 cells, indicating a high portion of leukemia cells existing in the blood, while the DsRed intensity for the mice challenged with LNT cells was similar to that of healthy mice (FIG. 1J, FIG. 1K).

### Example 3: Leveraging LNT Cells as the Targeting Drug Carrier

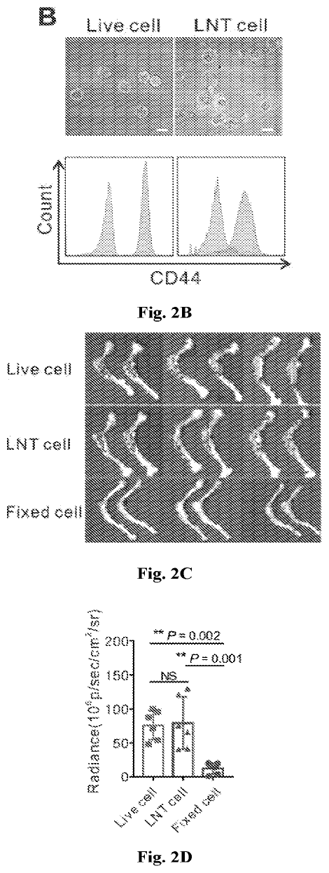

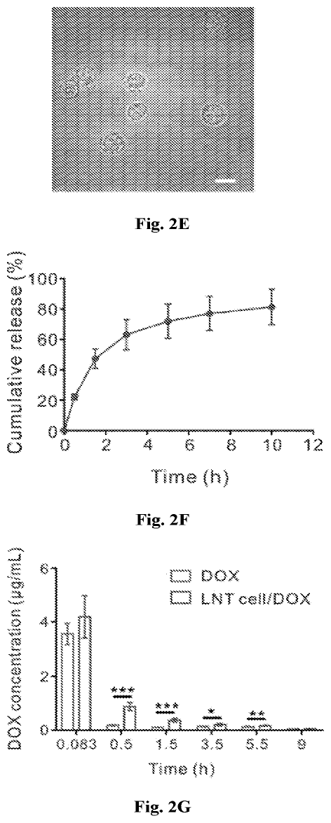

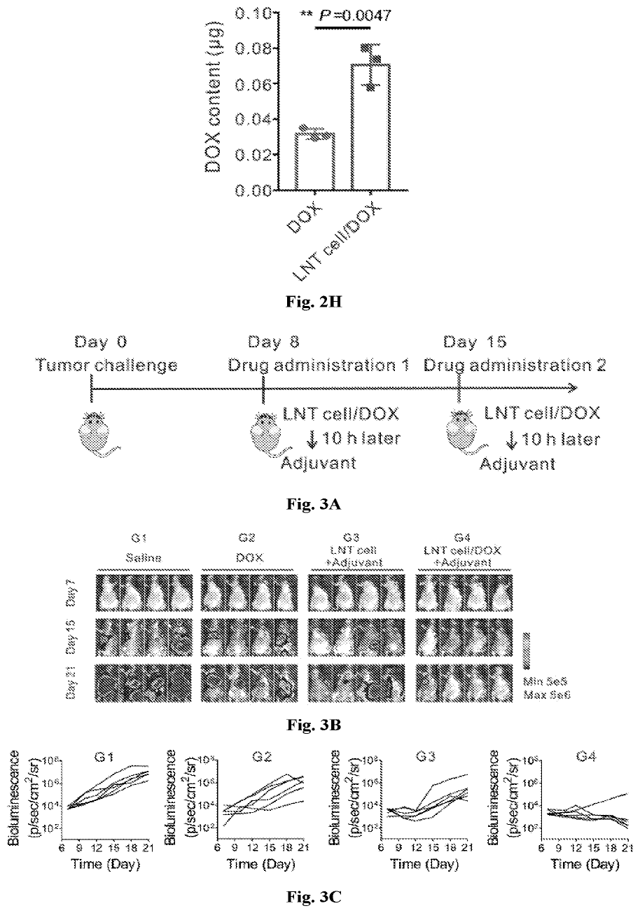

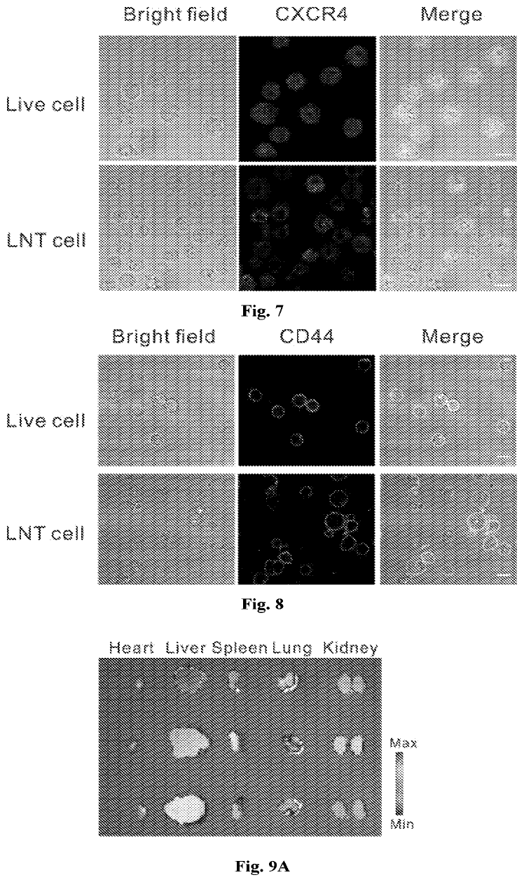

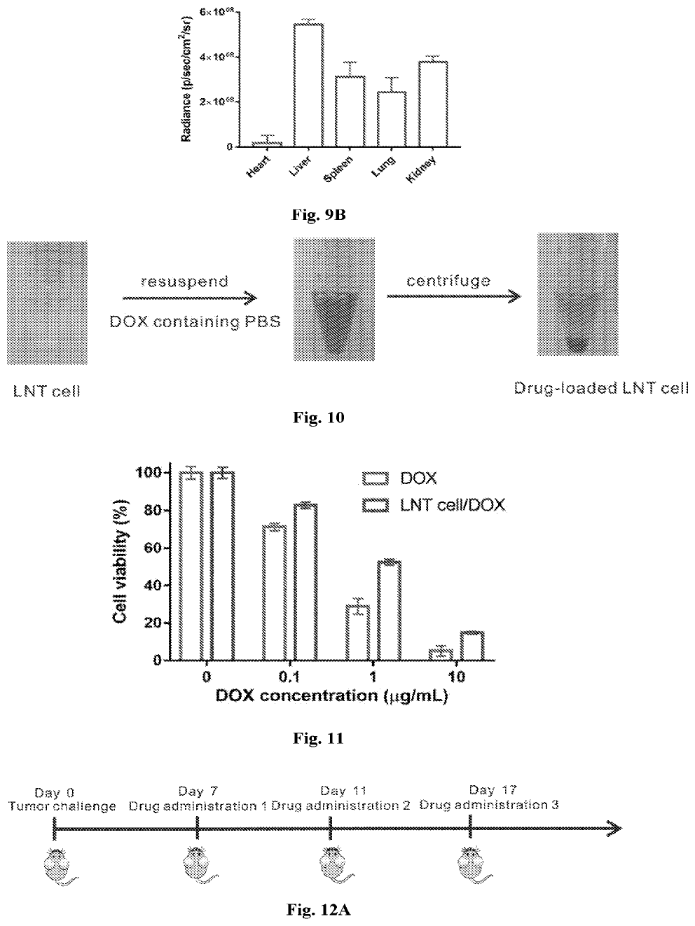

Leukemia cells exhibit bone marrow homing and resident capabilities, which are at least in part associated with the exhibition of CXCR4 and CD44 chemokine, two typical adhesion receptors that interact with bone marrow. SDS-PAGE (sodium dodecyl sulfate-polyacrylamide gel electrophoresis) showed that most of the proteins expressed by live C1498 cells were retained in LNT cells (FIG. 6). Of note, CXCR4 and CD44 were detected in both live cells and LNT cells as assessed by confocal imaging and flow cytometry (FIGS. 2A, 2B, 7, and 8). The bone marrow homing of LNT cells was also evaluated. Upon intravenous infusion, LNT cells exhibited similar accumulation efficiency in bone barrow in comparison to live C1498 cells. The cell signal was significantly higher than that of paraformaldehyde-fixed cells, which is likely due to the loss of bioactivities during paraformaldehyde fixation (FIGS. 2C, and 2D), indicating the bone marrow targeting capacity of AML cells was retained in LNT cells. LNT cells also distributed in liver, kidney and spleen, which are also infiltrating sites for AML cells, while seldom localized in the heart (FIG. 9).

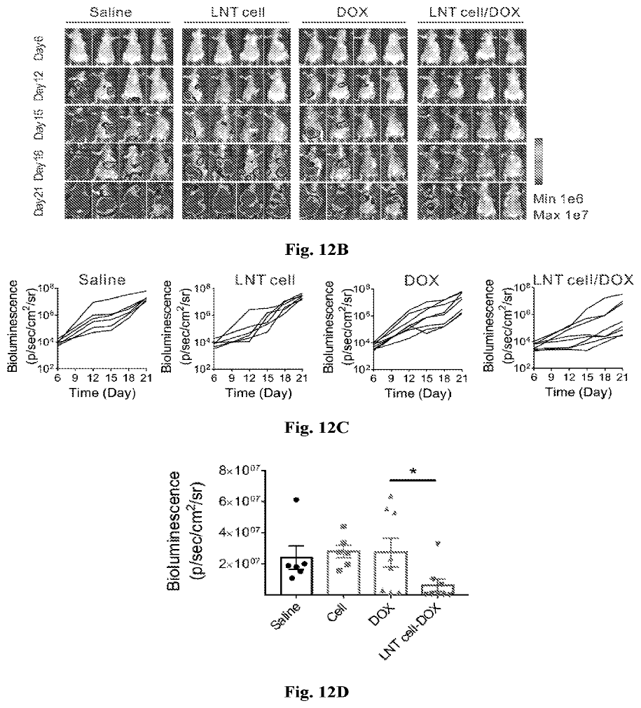

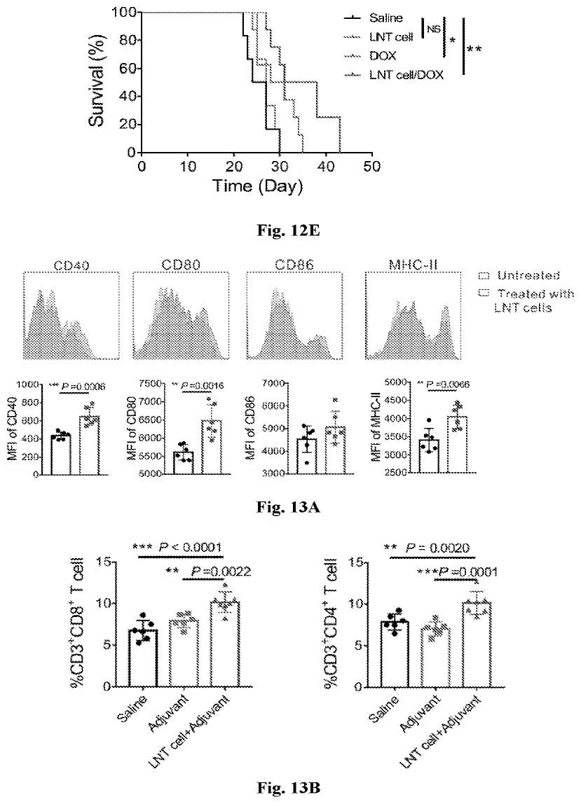

Since nuclear and cytoplasmic cellular structures are preserved in LNT cells (FIG. 1B), it is convenient to load the first-line anti-leukemia drug, doxorubicin (DOX), via DNA intercalation and the electrostatic interactions between DOX and cytoplasm proteins, and deliver DOX to bone marrow. Briefly, DOX can be loaded into LNT cells via mixing and incubation with a loading capacity of 65±16 μg of 1×10⁷ LNT cells (FIG. 2E, FIG. 10). DOX was released from the drug loaded LNT cells (LNT cell/DOX) in a sustained manner, and 81% of DOX was released within 10 h (FIG. 2F). We then studied the in vitro cytotoxicity against C1498 cells of free DOX and LNT cell/DOX. The IC₅₀ values were 0.32 μg/mL and 1.05 μg/mL, respectively (FIG. 11). Even though free DOX exhibited higher cytotoxicity against C1498 cells in vitro, LNT cell/DOX allowed significantly longer detection of DOX in the blood and higher DOX accumulation within the bone marrow (FIGS. 2G, and 2H). Murine AML models were utilized to evaluate the therapeutic efficacy of LNT cell/DOX. In tumor-bearing C57BL/6J mice, tumor growth was monitored by bioluminescence signals upon treatment (FIGS. 12A-12C). In this leukemia model, LNT cell/DOX treatment promoted better control of tumor growth compared to control treatments (FIGS. 12D, and 12E).

### Example 4: Chemo-Immunotherapy Via LNT Cells

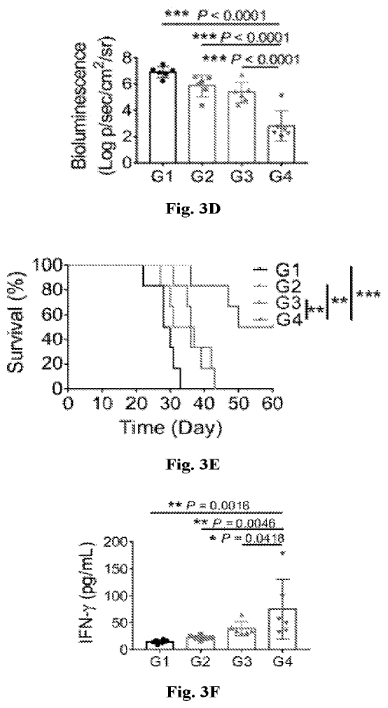

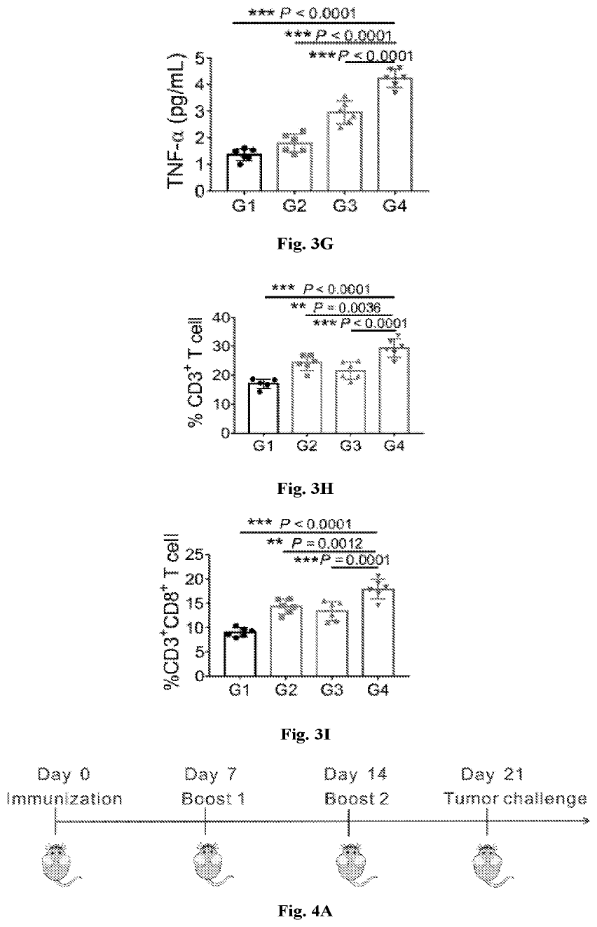

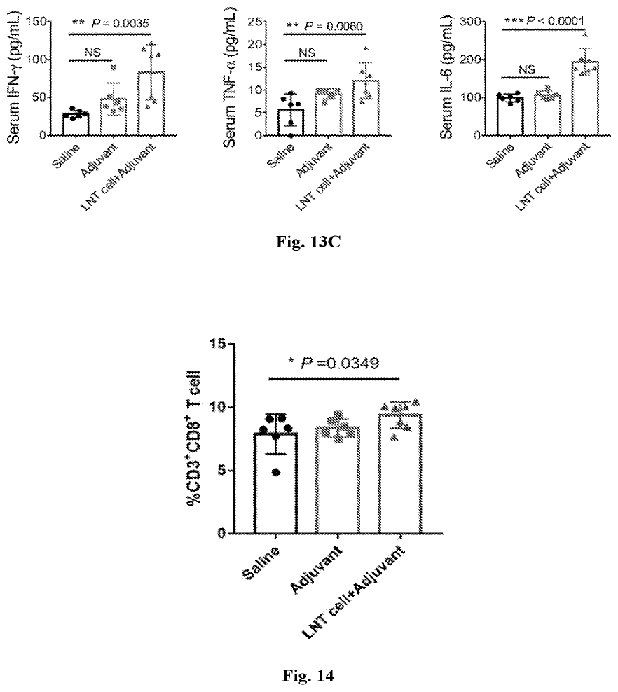

Tumor cell lysates can function as cancer vaccines and initiate tumor-specific immune responses. The LNT cells may enhance the antigen uptake and maturation of antigen presenting cells (APCs). LNT cells cocultured with dendritic cells (DCs) caused their maturation as assessed by upregulation of CD40, CD80, CD86 and MHC-II (FIG. 13A). Moreover, CD4⁺ T cells and CD8⁺ T cells increased in the peripheral blood of the mice receiving LNT cells and the adjuvant of monophosphoryl lipid A (MPLA) (FIG. 13B). DC maturation and T cell activation-related cytokines, including IFN-γ, TNF-α and IL-6, were also detected in mice treated with LNT cell and adjuvant (FIG. 13C). We next evaluated the anti-tumor efficacy of LNT cell/DOX with adjuvant in leukemia-bearing mice. As demonstrated in FIGS. 3A-3B, bioluminescence of AML cancer cells increased rapidly in untreated mice, while AML had been partially inhibited after DOX or LNT cell and adjuvant treatment. Remarkably, AML cells were almost completely eliminated in mice treated with LNT cell/DOX and adjuvant up to 21 days post-tumor inoculation (FIG. 3B). Quantitative analysis of tumor bioluminescence and survival analysis also demonstrated superior therapeutic activity of LNT cell/DOX combined with adjuvant (FIGS. 3C-3E). Increased serum levels of IFN-γ and TNF-α (FIGS. 3F, and 3G), as well as elevation of CD3⁺ T cell and CD8⁺ T cells supported the occurrence of boosted immunity in the mice receiving LNT Cell/DOX and adjuvant treatment (FIGS. 3H, and 3I).

### Example 4: Prophylactic Efficiency of LNT Tumor Cells

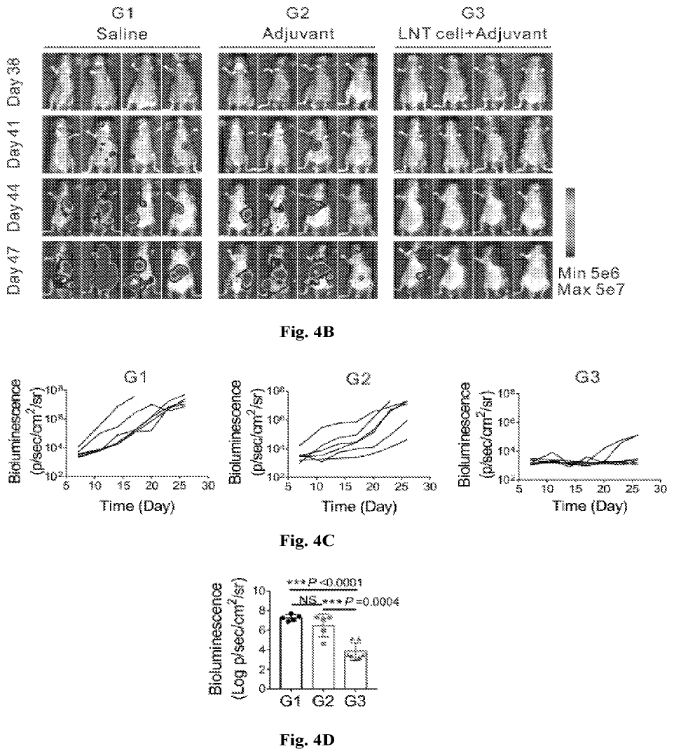

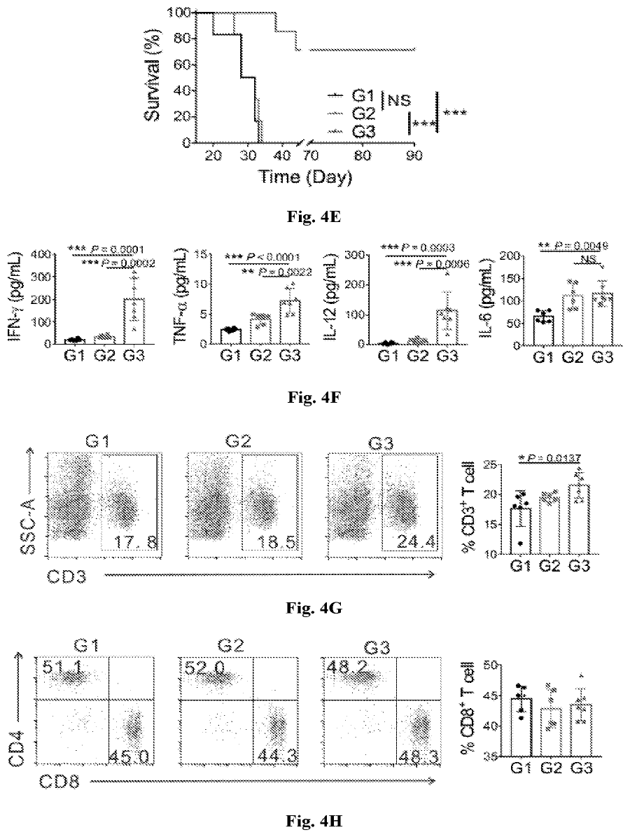

The efficacy of LNT cells was evaluated as a prophylactic cancer vaccine. Mice were first immunized at 21 days, 14 days and 7 days prior to challenge with live C1498 cells. The onset of AML in mice was significantly prevented in mice pre-immunized with LNT cells and adjuvant (FIGS. 4A-4C). Quantitative data also revealed that the tumor bioluminescence intensity of the group of LNT cells with adjuvant was significantly lower than control groups (FIG. 4D). Moreover, 71% of the mice treated with LNT cells and adjuvant were tumor free 90 days post tumor challenge, while all control mice died by day 34 (FIG. 4E). Serum levels of IFN-γ, TNF-α, IL-12 and IL-6 were significantly increased in mice treated with LNT cells and adjuvant (FIG. 4F), indicating that a prompt immune response was triggered upon tumor cell inoculation. In addition, CD3⁺ T cells and CD8⁺ T cells were significantly increased in the peripheral blood of mice vaccinated with LNT cells and adjuvant (FIGS. 4G, 4H, and 14).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition comprising dead cells, wherein the cells are cryo-shocked cells.

2. The composition of claim 1, wherein the cells are cryo-shocked in liquid nitrogen.

3. The composition of claim 1, wherein the cryo-shock eliminates the pathogenicity of the dead cells.

4. The composition of claim 1, wherein the dead cells maintain their major structure.

5. The composition of claim 1, wherein the dead cells maintain their chemotaxis towards a specific tissue.

6. The composition of claim 1, wherein the dead cells are loaded with a drug.

7. The composition of claim 6, wherein the drug is a cancer therapeutic.

8. The composition of claim 7, wherein the cancer therapeutic is a chemotherapeutic agent.

9. The composition of claim 8, wherein the chemotherapeutic agent is thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, calicheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid, or capecitabine.

10. The composition of claim 6, wherein the dead cells effect targeted delivery of the drug toward a target tissue.

11. The composition of claim 5, wherein the target tissue is epithelial tissue, connective tissue, bone marrow, or lymphatic system.

12. The composition of claim 1, wherein the dead cells are dead cancer cells.

13. The composition of claim 12, wherein the dead cancer cells promote an immune response.

14. The composition of claim 12, wherein the dead cancer cells activate maturation of dendritic cells.

15. The composition of claim 12, wherein the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

16. A vaccine comprising the composition of claim 1.

17. A method of treating or preventing cancer, comprising administering the composition of claim 1.

18. A method of treating or preventing cancer, comprising administering the vaccine of claim 16.

19-21. (canceled)

22. A method of preparing dead cryo-shocked cells, comprising shocking live cells in liquid nitrogen.

23-38. (canceled)

39. A method for delivering a drug to a target tissue of a patient comprising administering the pharmaceutical composition of claim 6.
